SEQUENCE LISTING (42 sequences)

SEQ ID NO: 1 - DNA, 19 bp, synthetic construct
  source 1..19: mol_type = other DNA
gaggccaagactgcgttcc

SEQ ID NO: 2 - DNA, 19 bp, synthetic construct
  source 1..19: mol_type = other DNA
ggtgtcatgggtcagcagc

SEQ ID NO: 3 - DNA, 20 bp, synthetic construct
  source 1..20: mol_type = other DNA
agtcggtgtatgccttctcg

SEQ ID NO: 4 - DNA, 20 bp, synthetic construct
  source 1..20: mol_type = other DNA
cgatgtcaggctcgatgtca

SEQ ID NO: 5 - DNA, 24 bp, synthetic construct
  source 1..24: mol_type = other DNA
ggagagacctttatgagaaagcaa

SEQ ID NO: 6 - DNA, 22 bp, synthetic construct
  source 1..22: mol_type = other DNA
gctgtcttcctggcatatcaca

SEQ ID NO: 7 - DNA, 20 bp, synthetic construct
  source 1..20: mol_type = other DNA
tgacggatgagcagtaccag

SEQ ID NO: 8 - DNA, 19 bp, synthetic construct
  source 1..19: mol_type = other DNA
gcttccgcatcttgcttgg

SEQ ID NO: 9 - DNA, 20 bp, synthetic construct
  source 1..20: mol_type = other DNA
tggacctctgccctctggat

SEQ ID NO: 10 - DNA, 20 bp, synthetic construct
  source 1..20: mol_type = other DNA
ggcagggaaccagcatcttc

SEQ ID NO: 11 - DNA, 20 bp, synthetic construct
  source 1..20: mol_type = other DNA
gagccaggtgtagtgtgtgc

SEQ ID NO: 12 - DNA, 20 bp, synthetic construct
  source 1..20: mol_type = other DNA
tcgtccacgtgtaagttgcg

SEQ ID NO: 13 - DNA, 20 bp, synthetic construct
  source 1..20: mol_type = other DNA
gggctttccgagatttgggg

SEQ ID NO: 14 - DNA, 20 bp, synthetic construct
  source 1..20: mol_type = other DNA
ccctcccagcacttgcattt

SEQ ID NO: 15 - DNA, 20 bp, synthetic construct
  source 1..20: mol_type = other DNA
ataggattgcccaggcagaa

SEQ ID NO: 16 - DNA, 25 bp, synthetic construct
  source 1..25: mol_type = other DNA
ctaagggtaaaagcagggagagagt

SEQ ID NO: 17 - DNA, 20 bp, synthetic construct
  source 1..20: mol_type = other DNA
ggccaggtcatcaccattgg

SEQ ID NO: 18 - DNA, 20 bp, synthetic construct
  source 1..20: mol_type = other DNA
ctttgcggatgtccacgtca

SEQ ID NO: 19 - DNA, 20 bp, synthetic construct
  source 1..20: mol_type = other DNA
gatcgcagcggtatgtgtcg

SEQ ID NO: 20 - DNA, 20 bp, synthetic construct
  source 1..20: mol_type = other DNA
ctgctgggacatcagttcgg

SEQ ID NO: 21 - DNA, 20 bp, synthetic construct
  source 1..20: mol_type = other DNA
gcgaaacaagcagaacccag

SEQ ID NO: 22 - DNA, 20 bp, synthetic construct
  source 1..20: mol_type = other DNA
gcctctggcgaagaaactct

SEQ ID NO: 23 - DNA, 20 bp, synthetic construct
  source 1..20: mol_type = other DNA
tttgcaccttcgtttcctgc

SEQ ID NO: 24 - DNA, 20 bp, synthetic construct
  source 1..20: mol_type = other DNA
tcccaatcacaccacaagcc

SEQ ID NO: 25 - DNA, 20 bp, synthetic construct
  source 1..20: mol_type = other DNA
catgcaaaccacacccacag

SEQ ID NO: 26 - DNA, 20 bp, synthetic construct
  source 1..20: mol_type = other DNA
tgagcactgaggacttgctg

SEQ ID NO: 27 - DNA, 20 bp, synthetic construct
  source 1..20: mol_type = other DNA
ccatcctggacgctcttgaa

SEQ ID NO: 28 - DNA, 20 bp, synthetic construct
  source 1..20: mol_type = other DNA
ggtctgtcaccaagtagggc

SEQ ID NO: 29 - DNA, 20 bp, synthetic construct
  source 1..20: mol_type = other DNA
tgtgatgaaagacggcacac

SEQ ID NO: 30 - DNA, 20 bp, synthetic construct
  source 1..20: mol_type = other DNA
tgtgcagactcaaactccac

SEQ ID NO: 31 - DNA, 20 bp, synthetic construct
  source 1..20: mol_type = other DNA
ggcagctaccaactgtgacc

SEQ ID NO: 32 - DNA, 20 bp, synthetic construct
  source 1..20: mol_type = other DNA
ctcccgacgacacactctct

SEQ ID NO: 33 - DNA, 20 bp, synthetic construct
  source 1..20: mol_type = other DNA
agctcaccaagctcctggat

SEQ ID NO: 34 - DNA, 20 bp, synthetic construct
  source 1..20: mol_type = other DNA
aagggaacaaggtgggtttg

SEQ ID NO: 35 - DNA, 20 bp, synthetic construct
  source 1..20: mol_type = other DNA
gaaccccacctgcttctacc

SEQ ID NO: 36 - DNA, 20 bp, synthetic construct
  source 1..20: mol_type = other DNA
gacattctgctgcatctgcg

SEQ ID NO: 37 - DNA, 20 bp, synthetic construct
  source 1..20: mol_type = other DNA
tgattctacccacggcaagt

SEQ ID NO: 38 - DNA, 20 bp, synthetic construct
  source 1..20: mol_type = other DNA
agcatcaccccatttgatgt

SEQ ID NO: 39 - RNA, 19 nt, synthetic construct
  source 1..19: mol_type = other RNA
cgtgacagtccttctggaa

SEQ ID NO: 40 - RNA, 19 nt, synthetic construct
  source 1..19: mol_type = other RNA
ttccagaaggactgtcacg

SEQ ID NO: 41 - RNA, 19 nt, synthetic construct
  source 1..19: mol_type = other RNA
ctctcttcacagccgaaga

SEQ ID NO: 42 - RNA, 19 nt, synthetic construct
  source 1..19: mol_type = other RNA
tcttcggctgtgaagagag

(19) United States
(12) Patent Application Publication
(10) Pub. No.: US 2025/0205253 A1
(43) Pub. Date: Jun. 26, 2025

(54) PHARMACEUTICAL COMPOSITION FOR PREVENTING OR TREATING BENIGN PROSTATIC HYPERPLASIA

(71) Applicant: University-Industry Cooperation Group of Kyung Hee University, Yongin-si (KR)

(72) Inventors: HYO-JIN AN, Seoul (KR); BO-RAM JIN, Seoul (KR)

(21) Appl. No.: 18/798,210

(22) Filed: Aug. 8, 2024

(30) Foreign Application Priority Data
Dec. 22, 2023 (KR) 10-2023-0189256

(51) Int. Cl. A61K 31/66; A61P 13/08

(52) U.S. Cl. CPC A61K 31/66; A61P 13/08

(57) ABSTRACT

Provided are a pharmaceutical composition, a health functional food, and a method for preventing or treating benign prostatic hyperplasia (BPH). The present invention includes mitoquinone as an active ingredient and inhibits the androgen receptor (AR) and nucleotide oligomerization domain (NOD)-like receptor family pyrin domain-containing 3 (NLRP3) signaling pathways to alleviate pathological prostatic hypertrophy and exhibit anti-proliferative and antioxidant effects. Therefore, it can be effectively used in preventing, treating, or ameliorating BPH.

### CROSS-REFERENCE TO RELATED APPLICATION

This application claims priority to and the benefit of Korean Patent Application No. 10-2023-0189256, filed on Dec. 22, 2023, the disclosure of which is incorporated herein by reference in its entirety.

### REFERENCE TO SEQUENCE LISTING SEQUENCE LISTING

This application contains a sequence listing that has been filed electronically in XML format, created on Jul. 22, 2024, and named “9-PJK4967239-SeqListing.xml” (38,020 bytes), the contents of which are incorporated herein by reference in their entirety.

### STATEMENT REGARDING PRIOR DISCLOSURE BY THE INVENTOR OR A JOINT INVENTOR

Applicant hereby states that non-patent literature titled “Antioxidant mitoquinone suppresses a benign prostatic hyperplasia by regulating the AR-NLRP3 pathway”, which is submitted with the application in an information disclosure statement under 37 CFR 1.97, is a prior disclosure made directly or indirectly by the inventor(s), and the prior disclosure made by the inventor(s) do not qualify as prior art under the grace period exception of 35 USC § 102 (b) (1).

### BACKGROUND

### 1. Field of the Invention

The present invention relates to a pharmaceutical composition and a health functional food for preventing or treating benign prostatic hyperplasia.

### 2. Discussion of Related Art

The prostate is one of the sexual appendages responsible for reproductive functions, along with the testes and seminal vesicles. The prostate produces about ⅓ of the liquid component of male semen, and the prostatic fluid not only provides nutrition to sperm produced in the testes, but also prevents solidification of ejaculated semen and thus increases sperm motility, thereby improving the sperm's fertility. In addition, because the prostatic fluid is alkaline, it serves as an important medium for sperm activity by neutralizing the strong acidity of the female fallopian tubes and helping sperm that reach the fallopian tubes to safely fertilize the egg.

Benign prostate hyperplasia (BPH) is a disease that is most common among men over 50 years of age, and it deteriorates quality of life by accompanying lower urinary tract symptoms. The biggest triggers for the development of BPH are increased age and the presence of male hormones. Men who have congenital testicular dysfunction or who have had their testes removed do not develop BPH, suggesting that male hormones are closely involved as a trigger. In addition, differences in race, environment, and dietary habits may also cause BPH.

Symptoms of BPH include difficulty in holding urine, thin and weak urine stream, hesitation for a long time even when feeling the urge to urinate, and a feeling of residual urine even after urinating. In particular, patients with BPH often feel the urge to urinate and thus have trouble sleeping. Because these symptoms progress gradually over a long period of time, it is easy to overlook them and dismiss them as simply symptoms of aging. As a result, there are cases where it develops into serious conditions such as hydronephrosis or uremia.

Meanwhile, treatments for BPH include drug therapy, non-invasive procedures, invasive surgery, and alternative treatments. Male hormones are deeply involved in the growth, development, and pathological conditions of the prostate through male hormone receptors in the prostate. BPH is determined by male hormones, and dihydrotestosterone (DHT) is the most important male hormone in the prostate, so it also plays an important role in BPH. Recently, herbal medicine treatments have been emerging to compensate for the side effects and shortcomings of existing synthetic drug treatments for BPH and male pattern baldness.

Accordingly, the present inventors conducted research to develop a composition having preventive or therapeutic activity against BPH using natural substances with guaranteed safety compared to synthetic chemicals. As a result, the present inventors found that mitoquinone has excellent preventive or therapeutic activity in an animal BPH model and completed the present invention.

### SUMMARY OF THE INVENTION

A technical problem to be achieved by the present invention is to provide a pharmaceutical composition for preventing or treating benign prostate hyperplasia (BPH).

Another technical problem to be achieved by the present invention is to provide a health functional food for preventing or ameliorating BPH.

The technical problem to be achieved by the present invention is not limited to the above-mentioned technical problems, and other technical problems that are not mentioned can be clearly understood by those skilled in the art from the description below.

To achieve the above technical problems, one embodiment of the present invention provides a pharmaceutical composition for preventing or treating BPH, including mitoquinone or a pharmaceutically acceptable salt thereof.

In an embodiment of the present invention, the mitoquinone may be included at 25 μM or more.

In an embodiment of the present invention, the mitoquinone may inhibit androgen receptor (AR) and nucleotide oligomerization domain (NOD)-like receptor family pyrin domain-containing 3 (NLRP3) signaling pathways.

In an embodiment of the present invention, the mitoquinone may inhibit dihydrotestosterone (DHT)-induced prostate cell proliferation.

To achieve the above technical problems, another embodiment of the present invention provides a health functional food for preventing or ameliorating BPH, including mitoquinone or a stiologically acceptable salt thereof.

### BRIEF DESCRIPTION OF THE DRAWINGS

FIGS. 1A-1G confirm an antioxidant effect of mitoquinone (MitoQ) in dihydrotestosterone (DHT)-stimulated RWPE-1 cells. (FIG. 1A) Molecular structure of MitoQ. (FIG. 1B) Viability of MitoQ-treated RWPE-1 cells at the indicated concentration for 24 h measured via the cell counting kit (CCK)-8 assay. (FIG. 1C) RWPE-1 cell proliferation under DHT (10 nM) stimulation and MitoQ at the indicated concentration for 24 h measured via the CCK-8 assay. (FIG. 1D) Expression of 8-hydroxy-2′-deoxyguanosine (8OHdG) in DHT-stimulated RWPE-1 cells treated with MitoQ (25, 50, and 100 μM). (FIG. 1E) The mRNA expression of heme oxygenase-1 (HO-1) and glutathione peroxidase 1 (GPX-1) in DHT-stimulated RWPE-1 cells treated with MitoQ (25, 50, and 100 μM). The results are expressed as means±SD (n=3). ^{###}p<0.001 vs. vehicle group; **p<0.01, ***p<0.001 vs. DHT-stimulated group. (FIG. 1F) Manifestation of MitoTracker™ and MitoSOX™ in DHT-stimulated MitoQ-treated RWPE-1 cells (100 μM); 0.6% H₂O₂ was used as positive control. (FIG. 1G) Catalase and superoxide dismutase 2 (SOD2) protein expression in DHT-stimulated RWPE-1 cells treated with MitoQ (25, 50, and 100 μM). The results are expressed as means±SD (n=3). ^{###}p<0.001 vs. vehicle group; **p<0.01, ***p<0.001 vs. DHT-stimulated group.

FIGS. 2A-2G show the effect of MitoQ on the nucleotide oligomerization domain (NOD)-like receptor family pyrin domain-containing 3 (NLRP3) signaling pathway in DHT-stimulated RWPE-1 cells. (FIG. 2A) NLRP3 expression in DHT-stimulated RWPE-1 cells treated with MitoQ (25, 50, and 100 μM). (FIG. 2B) The mRNA expression of NLRP3, PYCARD (pyrin domain and caspase activation and recruitment domain), and IL-1β in DHT-stimulated RWPE-1 cells treated with MitoQ (25, 50, and 100 μM). The results are expressed as means±SD (n=3). ^{###}p<0.001 vs. vehicle group; *p<0.05, **p<0.01, ***p<0.001 vs. DHT-stimulated group. (FIG. 2C) RWPE-1 cells were transfected with green fluorescent protein (GFP) or NLRP3 small interfering RNA (siRNA) and stimulated with or without DHT, and then transfected with NLRP3 siRNA. The mRNA expression of NLRP3 and IL-1β was estimated in transfected RWPE-1 cells. ^{###}p<0.001 vs. GFP group; ***p<0.001 vs. DHT-stimulated GFP group; ^{$$$}p<0.001 vs. DHT-stimulated NLRP3 siRNA-transfected group. (FIG. 2D) Molecular docking simulation on the NACHT domain of NLRP3 was conducted using Autodock Vina v1.1.2. (FIG. 2E) The ATPase activity in MitoQ-treated RWPE-1 cells was analyzed under DHT stimulation. (FIG. 2F) ATP level in DHT-stimulated RWPE-1 cells treated with MitoQ (25, 50, and 100 μM). The results are expressed as means±SD (n=3). ^{###}p<0.001 vs. vehicle group; ***p<0.001 vs. DHT-stimulated group. (FIG. 2G) NLRP3 and androgen receptor (AR) mRNA expression in NLRP3 siRNA-transfected or AR siRNA-transfected RWPE-1 cells. ^{###}p<0.001 compared with the GFP group; analysis of variance, followed by Dunnett's post-hoc test.

FIGS. 3A-3F show the inhibitory effect of MitoQ on AR-dependent cellular proliferation in DHT-stimulated RWPE-1 cells. (FIG. 3A) AR protein expression in DHT-stimulated RWPE-1 cells treated with MitoQ (25, 50, and 100 μM). (FIG. 3B) The mRNA levels of AR, 5α-reductase, and prostate-specific antigen (PSA) in DHT-stimulated RWPE-1 cells treated with MitoQ (25, 50, and 100 μM). (FIG. 3C) PSA protein level in DHT-stimulated RWPE-1 cells treated with MitoQ (25, 50, and 100 μM). (FIG. 3D) The mRNA expression of AR in NLRP3 siRNA-transfected RWPE-1 cells with or without 100 μM of MitoQ under DHT stimulation and (FIG. 3E) the mRNA expression of NLRP3 in AR siRNA-transfected RWPE-1 cells with or without 100 μM of MitoQ. The results are expressed as means±SD (n=3). ^{###}p<0.001 vs. GFP group; ***p<0.001 vs. DHT-stimulated GFP group; ^{$$$}p<0.001 vs. DHT-stimulated NLRP3 siRNA or AR siRNA-transfected group. (FIG. 3F) Molecular docking simulation of AR was conducted using Autodock Vina v1.1.2.

FIGS. 4A-4F show the effect of MitoQ on prostate cell proliferation in rats with BPH. (FIG. 4A) Representative prostate pictures from each experimental group. (FIG. 4B) Prostate weight to body weight (PW/BW) ratio was calculated at the end of the experiments. (FIG. 4C) Haemotoxylin and eosin (H&E) and immunohistochemistry (IHC) staining for proliferating cell nuclear antigen (PCNA) were conducted using prostate tissue sections. Images from each group were observed using a Leica microscope (original magnification=×40). (FIG. 4D) The thickness of the epithelium of the prostate tissue was measured using Leica Application Suite software. (FIG. 4E) The extent of PCNA-positive units was measured based on IHC staining. (FIG. 4F) PCNA mRNA level in the prostate of each experimental group was quantified using quantitative reverse transcription polymerase chain reaction (qRT-PCR). ^{###}p<0.001 compared with the Con group; ***p<0.001 compared with the BPH groups; analysis of variance, followed by Dunnett's post-hoc test.

FIGS. 5A-5E show the effect of MitoQ on oxidative stress in rats with BPH. (FIG. 5A) An immunofluorescence (IF) assay was conducted to detect the protein expression of (8-OHdG). (FIG. 5B) The extent of 8-OHdG positive unit was measured based on the IF assay. (FIG. 5C) The reactive oxygen species (ROS) scavenging effect of MitoQ was measured using an ROS assay. The extent of ROS positive cells from prostate was calculated. (FIG. 5D) The mRNA expression of HO-1, superoxide dismutase 1 (SOD1), and peroxisome proliferator-activated receptor gamma coactivator 1α (PGC1α) was evaluated using qRT-PCR. ^{###}P<0.001 compared with the Con group; ***P<0.001 compared with the BPH groups; analysis of variance, followed by Dunnett's post-hoc test. (FIG. 5F) The protein expression of catalase and SOD2 in rats with BPH. The results are expressed as the mean±SD (n=3). ^{###}p<0.001 vs. vehicle group; ***p<0.001 compared with the BPH groups.

FIGS. 6A-6E show the effect of MitoQ on NLPR3 signaling pathway in rats with BPH. (FIG. 6A) An IF assay was conducted to detect NLRP3 protein expression. (FIG. 6B) NLRP3-positive units were measured based on the IF assay. (FIG. 6C) The mRNA expression of PYCARD and IL-1β was evaluated using qRT-PCR. (FIG. 6D) The protein level of NLRP3, pro-caspase 1, cleaved caspase 1, and IL-1β was measured using western blotting. (FIG. 6E) The relative protein level was normalized to that of β-actin (housekeeping gene). ^{###}p<0.001 compared with the Con group; *p<0.05, **p<0.01, and ***p<0.001 compared with the BPH groups; analysis of variance, followed by Dunnett's post-hoc test.

FIGS. 7A-7E show the effect of MitoQ on the androgen/AR signaling pathway in rats with BPH. (FIG. 7A) DHT level in the serum from each experimental group was measured using an enzyme-linked immunosorbent assay (ELISA) kit. (FIGS. 7B and 7C) The mRNA level of (FIG. 7B) 5α-reductase 2 and (FIG. 7C) AR and steroid receptor coactivator-1 (SRC-1) was quantified using qRT-PCR. (FIG. 7D) PSA protein expression was evaluated using western blotting. **p<0.01, ***p<0.001 compared with the Con group; **p<0.01, ***p<0.001 compared with the BPH groups; analysis of variance, followed by Dunnett's post-hoc test. (FIG. 7E) Testosterone level in rats with BPH. The level of testosterone in the serum from each experimental group was measured using an ELISA kit.

### DETAILED DESCRIPTION OF EXEMPLARY EMBODIMENTS

Hereinafter, the present invention will be described in detail.

The present invention relates to a pharmaceutical composition for preventing or treating BPH.

The present invention includes mitoquinone or a pharmaceutically acceptable salt thereof.

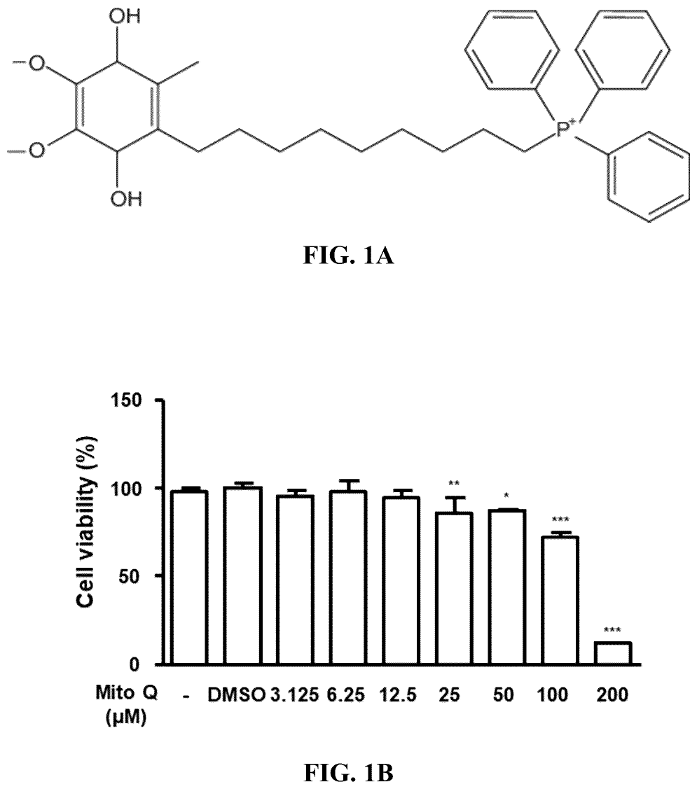

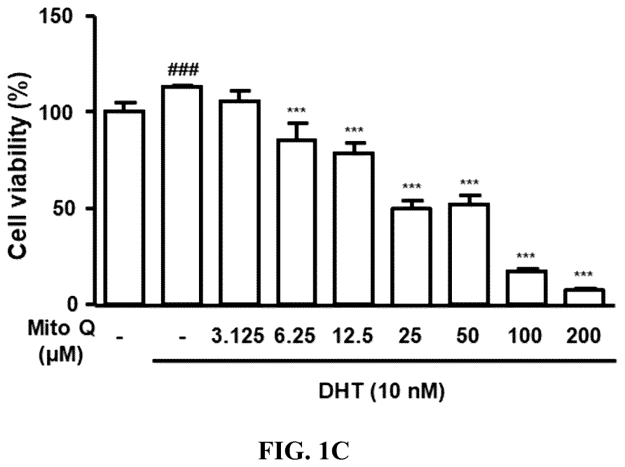

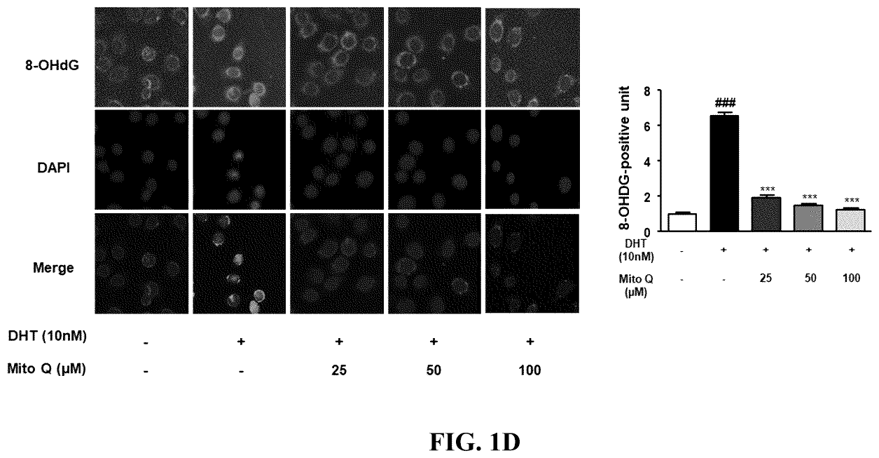

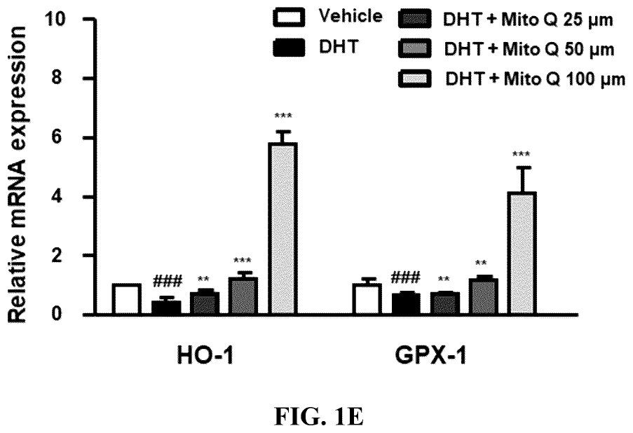

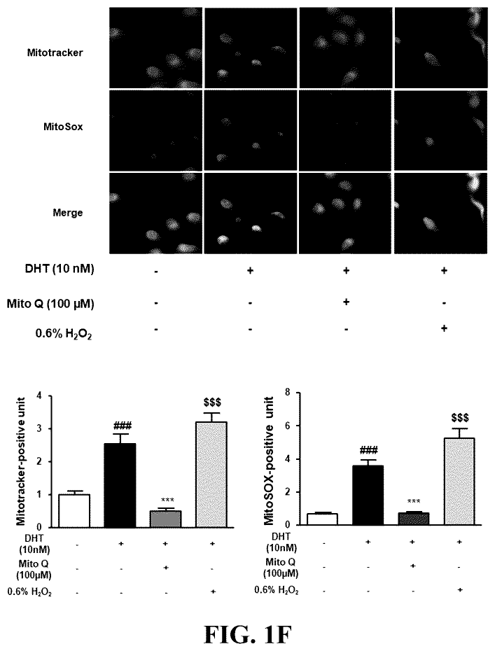

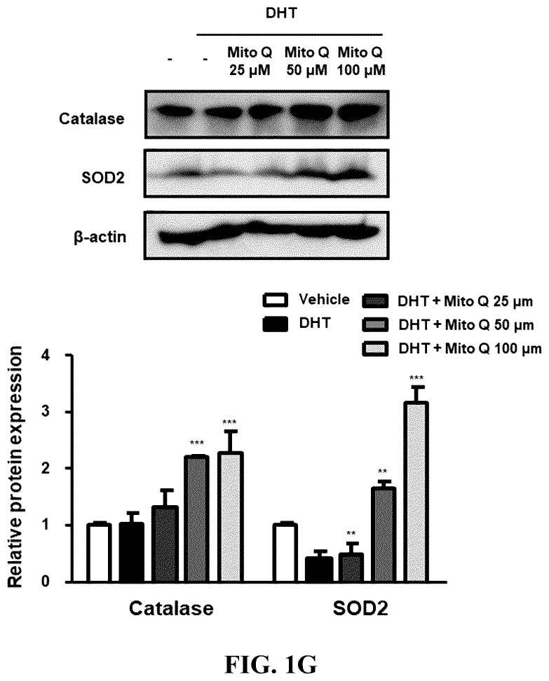

The mitoquinone is a compound represented by the following Chemical Formula 1 (FIG. 1A).

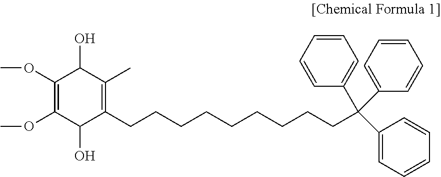 [SMILES: COC1=C(OC)C(O)C(CCCCCCCCCC(c2ccccc2)(c2ccccc2)c2ccccc2)=C(C)C1O]

As used herein, the term “pharmaceutically acceptable” refers to a compound, material, composition and/or dosage form that are suitable for use in contact with a human tissue within the scope of sound medical judgment, free from undue toxicity, irritation, allergic reaction, or other problems or complications, and commensurate with a reasonable benefit/risk ratio.

The “pharmaceutically acceptable salt” refers to a derivative of a disclosed compound in which the parent compound has been modified by preparing an acid or base salt thereof. Examples may include inorganic or organic acid salts of basic moieties such as amines, and alkaline or organic salts of acidic moieties such as carboxylic acids, but are not limited thereto. For example, conventional non-toxic salts or quaternary ammonium salts of a parent compound formed from non-toxic inorganic or organic acids may be included. For example, such conventional non-toxic salts may include salts derived from inorganic acids such as hydrochloric acid, hydrobromic acid, sulfuric acid, sulfamic acid, phosphoric acid, nitric acid, and the like; and salts derived from organic acids such as acetic acid, propionic acid, succinic acid, glycolic acid, stearic acid, lactic acid, malic acid, tartaric acid, citric acid, ascorbic acid, pamoic acid, maleic acid, hydroxymaleic acid, phenylacetic acid, glutamic acid, benzoic acid, salicylic acid, sulfanilic acid, 2-acetoxybenzoic acid, fumaric acid, toluenesulfonic acid, methanesulfonic acid, ethane disulfonic acid, oxalic acid, isethionic acid, and the like.

A pharmaceutically acceptable salt of the compound may be synthesized from a parent compound containing a basic or acidic moiety by a conventional chemical method. In general, such a salt may be prepared by subjecting a free acid or base form of a compound to a reaction with a stoichiometric amount of an appropriate base or acid in water or an organic solvent or mixtures thereof. Generally, non-aqueous media such as ether, ethyl acetate, ethanol, isopropanol or acetonitrile are preferred. A list of suitable salts is found in Remington's Pharmaceutical Sciences, 17th ed., Mack Publishing Company, Easton, PA, 1985, p. 1418, the disclosed content of which is incorporated herein by reference in its entirety.

In addition, the composition of the present invention may include not only pharmaceutically acceptable salts of mitoquinone, but also all possible solvates and hydrates that may be prepared therefrom, and may also include all possible stereoisomers.

The mitoquinone may be included, for example, in an amount of 25 μM or more, 50 μM or more, or 100 μM or more. Although not limited to the above range, when the mitoquinone is included in the composition at the above concentration, the effect of preventing, treating or ameliorating BPH may be maximized by inhibiting prostate cell proliferation and mitochondrial ROS production.

In addition, the mitoquinone may participate in the AR and NLRP3 signaling pathways and downregulate AR and NLRP3, thereby inhibiting DHT-induced prostate cell proliferation.

The pharmaceutical composition of the present invention further includes a

pharmaceutically acceptable carrier, which is commonly used in preparation, such as lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, and mineral oils, but is not limited thereto.

In addition to the above ingredients, the pharmaceutical composition of the present invention may further include lubricants, wetting agents, sweeteners, flavoring agents, emulsifiers, suspending agents, preservatives, and the like. Suitable pharmaceutically acceptable carriers and formulations are described in detail in Remington's Pharmaceutical Sciences (19th ed., 1995). The appropriate dose of the pharmaceutical composition of the present invention varies depending on factors such as formulation method, administration method, the patient's age, body weight, and sex, severity of disease symptoms, food, administration time, administration route, excretion rate, and reaction sensitivity, and a doctor of ordinary skill may easily determine and prescribe an effective doss for the desired treatment. Meanwhile, the dose of the pharmaceutical composition of the present invention may be 0.01 to 2000 mg/kg (body weight) per day, but is not limited thereto.

The pharmaceutical composition of the present invention may be administered orally or parenterally, and when administered parenterally, it may be administered by intravenous injection, subcutaneous injection, intramuscular injection, intraperitoneal injection, transdermal administration, and the like. The administration route of the pharmaceutical composition of the present invention may preferably be determined depending on the type of disease to which it is applied.

The pharmaceutical composition of the present invention may be prepared in the form of a unit dosage or it may be prepared by placing into a multi-dose container, as it is formulated using a pharmaceutically acceptable carrier and/or excipient according to a method that may be easily implemented by a person skilled in the art to which the present invention pertains. At this time, the formulation may be in the form of a solution, suspension or emulsion in an oil or an aqueous medium, or it may be in the form of an extract, powder, granules, tablets or capsules, and may further contain a dispersant and/or stabilizer.

The present invention relates to a health functional food for preventing or ameliorating BPH.

The present invention includes mitoquinone or a stiologically acceptable salt thereof.

The compounds included in the health functional food of the present invention, the action mechanisms thereof, and the effects thereof are as described above.

The health functional food of the present invention may be prepared and processed in the form of tablets, capsules, powder, granules, liquids, pills, and the like for the purpose of preventing or ameliorating BPH.

The health functional food of the present invention refers to food manufactured and processed using raw materials or ingredients with functional properties useful to the human body, and food that is taken in for a purpose of adjusting nutrients to the structure and function of the human body or obtaining useful effects in a hygienic use such as physiological effects.

The health functional food of the present invention may include common food additives, and its suitability as a food additive is determined by the specifications and standards for the item according to the general provisions and general test methods of the Food Additives Code approved by the Food and Drug Administration, unless otherwise specified.

The items listed in the Food Additives Code include, for example, chemical compounds such as ketones, glycine, calcium citrate, nicotinic acid, and cinnamic acid; natural additives such as persimmon color, crystalline cellulose, Kaoliang color, and guar gum; and mixed preparations such as sodium L-glutamate preparations, noodle-added alkaline preparations, preservative preparations, and tar coloring preparations, but are not limited thereto.

For example, for manufacture of health functional food in the form of tablets, a mixture prepared by mixing the compound with an excipient, a binder, a disintegrant, and other additives may be granulated by a conventional method and then molded by compression after adding a lubricant, or the mixture may be directly molded by compression. In addition, the health functional food in the form of tablets may contain a flavor enhancer and the like as needed.

Among the health functional food in the form of capsules, hard capsules may be manufactured by filling common hard capsules with a mixture prepared by mixing the peptide with additives such as excipients, and soft capsules may be manufactured by filling a mixture prepared by mixing the peptide with additives such as excipients into a capsule base such as gelatin. The soft capsules may contain plasticizers such as glycerin or sorbitol, colorants, preservatives, and the like as needed.

The health functional food in the form of pills may be prepared by molding a mixture prepared by mixing the compound with an excipient, a binder, a disintegrant, and the like by a known method, and it may be coated with white sugar or another coating agent or the surface may be coated with a material such as starch and talc as needed.

The health functional food in the form of granules may be manufactured into granules by mixing the compound with an excipient, a binder, a disintegrant, and the like by a conventional method, and it may contain a flavoring agent, a flavor enhancer, etc. as needed.

The health functional food includes beverages, meat, chocolate, food, confectioneries, pizza, ramen, other noodles, gum, candies, ice cream, alcoholic beverages, vitamin supplements, and health supplements.

The above health functional food may be applied orally as a nutritional supplement, and the form of application is not particularly limited.

The present invention relates to a method of treating BPH.

The treatment method of the present invention includes administering mitoquinone or a pharmaceutically acceptable salt thereof or a pharmaceutical composition including the same to an individual.

The mitoquinone or pharmaceutically acceptable salt thereof or pharmaceutical composition are as described above.

The individual refers to a subject in need of treatment for a disease, and more specifically, it may refer to mammals such as human or non-human primates, mice, rats, dogs, cats, horses, and cows.

The administering may include parenteral administration or oral administration, and methods for these administrations are known to those skilled in the art, and specific examples are as described above.

Hereinafter, the present invention will be described in detail with reference to examples.

### Materials and Methods

### 1. Cell Culture and Sample Treatment

The normal human prostate epithelial cell line, RWPE-1, was obtained from the American Type Culture Collection (Manassas, VA, USA).

The RWPE-1 cells were cultured in a keratinocyte serum-free medium supplemented with 0.05 mg/mL bovine pituitary extract, 5 ng/mL human recombinant epidermal growth factor, and antibiotic-antimycotic solution (Gibco®, Big Cabin, OK, USA). The cells were cultured in a humidified environment at 37° C. and 5% CO₂. The cells were cultured in a serum-free medium to block the effects of autocrine androgens. To mimic BPH induced by androgens in vivo, seeded cells were treated with 10 nM DHT for 72 h with or without various concentrations of MitoQ (25, 50, and 100 μM).

### 2. CCK-8 Assay

The cell viability and proliferation were measured using the Cell Counting Kit-8 (CCK-8, Dojindo Molecular Technologies, Inc., D.C., USA). The RWPE-1 cells were plated to 96-well plates (1×10⁵ cells/well) and incubated for 24 h. The following day, the cells were pretreated with or without various concentrations of MitoQ (3.125 to 200 μM) for one hour and then stimulated with 10 nM DHT for 24 h. After the treatment, the CCK-8 solution was added to each well and incubated for four hours. The number of viable cells was monitored by measuring absorbance at 450 nm using an Epoch microplate reader (Biotek, Winooski, VT, USA).

### 3. Immunofluorescence (IF)

The RWPE-1 cells treated with DHT and/or MitoQ (25, 50, and 100 μM) were fixed in 100% methanol, blocked with 10% normal goat serum (Gibco®), and treated with 8-OHdG (Cat. No. sc-66036; Santa Cruz Biotechnology), NLRP3 (Cat. No. NBP2-12446, Novus Biologicals), and AR (Cat. No. sc-7305) overnight. Thereafter, the cells were washed and incubated with fluorescein isothiocyanate (FITC)-conjugated anti-mouse or tetramethylrhodamine (TRITC)-conjugated anti-rabbit secondary antibodies. Nuclei were stained with 4′,6-diamidino-2-phenylindole (Life Technologies). Images were captured using an optical microscope (ECLIPSE Ni-U, Nikon, Tokyo, Japan).

After deparaffinization and rehydration, rat prostate tissue slides were incubated with anti-mouse 8-OHdG antibodies or anti-rabbit NLRP3 antibodies and visualized with FITC-conjugated anti-mouse and TRITC-conjugated anti-rabbit secondary antibodies, respectively.

The slides were mounted and detected using a Nikon X-Cite-Series 120 Q microscope (Nikon, Japan). The exposure parameters were the same for each sample.

4. Assessment of Mitochondrial Activity and Mitochondrial Reactive Oxygen Species (mtROS) Levels

The RWPE-1 cells were plated on a chamber slide at a density of 1×10⁵ cells/well the day before treatment. The RWPE-1 cells were treated with 10 nM DHT and/or 100 μM MitoQ for 24 h. Before incubating with a fluorescent dye, the RWPE-1 cells were treated with 0.6% H₂O₂ for 30 minutes. Mitochondrial activity and mtROS levels were measured by incubating the cells with MitoTracker™ Green FM and MitoSOX™ Mitochondrial Superoxide Indicator (Invitrogen, MA, USA) for 30 minutes at 37° C. The slides were mounted and detected using an EVOS M5000 microscope (Invitrogen).

### 5. Western Blot Analysis

Total protein was extracted from the RWPE-1 cells and rat prostate tissues, and a western blot analysis was performed. An antibody against AR (Cat. No. sc-7305) was purchased from Santa Cruz Biotechnology, Inc. (TX, USA). Boster Biological Technology (CA, USA) provided an antibody against a prostate-specific antigen (PSA; Cat. No. PB9259). An antibody against caspase-3 (Cat. No. #9661) was purchased from Cell Signaling Technology. Novus Biologicals (CO, USA) provided an antibody against NLRP3 (Cat. No. NBP2-12446). An antibody against IL-1β (Cat. No. 2105) was purchased from Abcam (Cambridge, UK).

6. Quantitative Real-Time Polymerase Chain Reaction (qRT-PCR) Analysis

Total RNA was isolated using Easy-Blue® reagent (iNtRON Biotechnology, Inc., Gyeonggi, Korea) and transcribed into complementary DNA (cDNA) according to the manufacturer's instructions. Oligonucleotide primers (Table 1) were purchased from Bioneer Corporation (Daejeon, Korea). qRT-PCR was performed according to the known method.

TABLE 1
Primer Sequences

| Gene name | Sense primers | Anti-sense primers |
|---|---|---|
| Human | GAGGCCAAGACTGCGTTCC | GGTGTCATGGGTCAGCAGC |
| HO-1 | (SEQ ID NO.: 1) | (SEQ ID NO.: 2) |
| Human | AGTCGGTGTATGCCTTCTCG | CGATGTCAGGCTCGATGTCA |
| GPX-1 | (SEQ ID NO.: 3) | (SEQ ID NO.: 4) |
| Human | GGAGAGACCTTTATGAGAAAGCAA | GCTGTCTTCCTGGCATATCACA |
| NLRP3 | (SEQ ID NO.: 5) | (SEQ ID NO.: 6) |
| Human | TGACGGATGAGCAGTACCAG | GCTTCCGCATCTTGCTTGG |
| PYCARD | (SEQ ID NO.: 7) | (SEQ ID NO.: 8) |
| Human | TGGACCTCTGCCCTCTGGAT | GGCAGGGAACCAGCATCTTC |
| IL-1ß | (SEQ ID NO.: 9) | (SEQ ID NO.: 10) |
| Human | GAGCCAGGTGTAGTGTGTGC | TCGTCCACGTGTAAGTTGCG |
| AR | (SEQ ID NO.: 11) | (SEQ ID NO.: 12) |
| Human | GGGCTTTCCGAGATTTGGGG | CCCTCCCAGCACTTGCATTT |
| 5α-reductase 2 | (SEQ ID NO.: 13) | (SEQ ID NO.: 14) |
| Human | ATAGGATTGCCCAGGCAGAA | CTAAGGGTAAAAGCAGGGAGAGAGT |
| PSA | (SEQ ID NO.: 15) | (SEQ ID NO.: 16) |
| Human | GGCCAGGTCATCACCATTGG | CTTTGCGGATGTCCACGTCA |
| ß-actin | (SEQ ID NO.: 17) | (SEQ ID NO.: 18) |
| Rat | GATCGCAGCGGTATGTGTCG | CTGCTGGGACATCAGTTCGG |
| PCNA | (SEQ ID NO.: 19) | (SEQ ID NO.: 20) |
| Rat HO-1 | GCGAAACAAGCAGAACCCAG | GCCTCTGGCGAAGAAACTCT |
| | (SEQ ID NO.: 21) | (SEQ ID NO.: 22) |
| Rat SOD-1 | TTTGCACCTTCGTTTCCTGC | TCCCAATCACACCACAAGCC |
| | (SEQ ID NO.: 23) | (SEQ ID NO.: 24) |
| Rat | CATGCAAACCACACCCACAG | TGAGCACTGAGGACTTGCTG |
| PGC1α | (SEQ ID NO.: 25) | (SEQ ID NO.: 26) |
| Rat | CCATCCTGGACGCTCTTGAA | GGTCTGTCACCAAGTAGGGC |
| PYCARD | (SEQ ID NO.: 27) | (SEQ ID NO.: 28) |
| Rat IL-1ß | TGTGATGAAAGACGGCACAC | TGTGCAGACTCAAACTCCAC |
| | (SEQ ID NO.: 29) | (SEQ ID NO.: 30) |
| Rat 5α- | GGCAGCTACCAACTGTGACC | CTCCCGACGACACACTCTCT |
| reductase 2 | (SEQ ID NO.: 31) | (SEQ ID NO.: 32) |
| Rat AR | AGCTCACCAAGCTCCTGGAT | AAGGGAACAAGGTGGGTTTG |
| | (SEQ ID NO.: 33) | (SEQ ID NO.: 34) |
| Rat SRC-1 | GAACCCCACCTGCTTCTACC | GACATTCTGCTGCATCTGCG |
| | (SEQ ID NO.: 35) | (SEQ ID NO.: 36) |
| Rat | TGATTCTACCCACGGCAAGT | AGCATCACCCCATTTGATGT |
| GAPDH | (SEQ ID NO.: 37) | (SEQ ID NO.: 38) |

7. Transfection of siRNA

The RWPE-1 cells were transfected with siRNA (Bioneer Corporation) or pmaxGFP™ vector using a 4D-Nucleofector™ system (Lonza, Basel, Switzerland). The following siRNA nucleotide sequences were used: NLRP3 siRNA, CGUGACAGUCCUUCUGGAA=tt(1-AS) (SEQ ID NO.: 39) and UUCCAGAAGGACUGUCACG=tt (1-AA) (SEQ ID NO.: 40) and AR siRNA, CUCUCUUCACAGCCGAAGA=tt (6-AS) (forward) (SEQ ID NO.: 41) and UCUUCGGCUGUGAAGAGAG=tt (6-AA) (reverse) (SEQ ID NO.: 42). After four hours of incubation, the cells were treated in the presence or absence of MitoQ 100 μM. The effect of NLRP3 or AR knockdown was detected using qRT-PCR.

### 8. Docking Simulation

A crystal structure of the NACHT domain of NLRP3 was recently identified. The structure was downloaded from the Protein Data Bank (PDB ID: 7ALV). This structure contains a potent di-aryl sulfonylurea inhibitor, RM5 (PubChem CID: 10195003). For AR (PDB ID: 2AMA), a crystal structure to which DHT is bound was used. The coordinates and size of the search space for the docking simulation were determined based on the 3D coordinate ranges of RM5 in the NACHT domain and the DHT binding site of the AR. A docking simulation was performed using AutoDock Vina v1.1.2 for testosterone, DHT, MitoQ, and RM5. The binding affinity of each ligand was predicted based on the best mode of binding, and the corresponding binding energy was calculated in kcal/mol. A lower binding energy indicates a greater binding affinity.

### 9. ATPase Assay

The RWPE-1 cells were seeded in dishes at a density of 1×10⁵ cells/well, and a serum-free medium was replaced the next day. The RWPE-1 cells were treated with 10 nM DHT and/or MitoQ (25, 50, and 100 μM) for 24 h. The ATPase activity was determined using a commercially available kit (Abcam, MA, USA). According to the manufacturer's instructions, the cells were homogenized in an ATPase assay buffer and centrifuged at 4° C. for ten minutes, and the supernatant was used. Phosphate in the cell lysate was removed using an ammonium sulfate solution (Thermo Fisher, MA, USA). The reaction mixture and ATPase assay developer were added to the samples, and ATPase activity was measured at 650 nm using an Epoch microplate spectrophotometer (BioTek, Winooski, VT, USA).

### 10. ATP Determination Assay

The RWPE-1 cells were seeded in dishes at a density of 1×10⁵ cells/well and treated with 10 nM DHT and/or MitoQ (25, 50, and 100 μM) for 24 h. The ATP level was determined using a CellTiter-Glo® Luminescent Cell Viability Assay kit (Promega, WI, USA). ATP disodium salt (Sigma Aldrich, St. Louis, MI, USA) was used to generate an ATP standard curve.

CellTiter-Glo® reagent was added to the samples, and luminescence was measured using an Epoch microplate spectrophotometer (BioTek, Winooski, VT, USA).

11. Experimental Animals and Sample treatment

Six-week-old male Sprague-Dawley rats (n=40; 200 +20 g) were purchased from Daehan Biolink Co. (Daejeon, Korea). The animals were randomly allocated, four rats in each cage, and housed in accordance with the guidelines for the care and use of laboratory animals adopted and promulgated by Sangji University according to the requirements established by the National Institutes of Health. All experimental protocols were approved by the Institutional Animal Care and Use Committee (IACUC) of Sangji University before the initiation of the study (IACUC Animal Approval Protocol #2018-18). The rats were acclimatized to laboratory conditions for one week before initiating the experiment. Briefly, the rats were randomly divided into five groups (n=8 per group): group 1, control (Con, normal prostate with vehicle: 100 μL corn oil, s.c.); group 2, BPH-induced rats (BPH, 10 mg/kg testosterone propionate dissolved in corn oil); group 3, BPH-induced rats treated with saw palmetto, 100 mg/kg/day, p.o. (Saw); group 4, BPH-induced rats treated with MitoQ, 5 mg/kg/day, p.o. (Mito 5); and group 5, BPH-induced rats treated with MitoQ, 25 mg/kg/day, p.o. (Mito 25). To eliminate the influence of intrinsic testosterone, all rats in the BPH-induced groups underwent bilateral orchiectomy, which was performed seven days prior to the administration of testosterone propionate.

The rats in the control group were incised and sutured without removing the testicles following anesthesia using zoletil 50 (intraperitoneal injection, 10 mg/kg). After a recovery period of one week, the rats in the BPH-induced groups were subcutaneously injected with 10 mg/kg/day testosterone propionate alone or in combination with MitoQ or Saw for four weeks, except on weekends. Twenty-four hours after the final administration, all rats were euthanized following anesthesia with zoletil 50 (i.p., 20 mg/kg). Blood samples were drawn through cardiac puncture, and serum was obtained via centrifugation and stored at −80° C.

### 12. Histological Analysis

Prostate tissues from each group were fixed in 4% formalin and embedded in paraffin. The tissues were cut into 4-μm sections. The sections were stained with H&E for histological examination. Images were acquired using a Leica DFC 295 microscope (Leica, Wetzlar, Germany). The thickness of the epithelium in the prostate tissues was measured using the Leica Application Suite software (LAS ver. 3.3.0, Leica Microsystems, Inc., Buffalo Grove, IL, USA).

### 13. Immunohistochemistry (IHC)

IHC was performed using the formalin-fixed paraffin-embedded samples. The paraffin blocks were cut into 4-μm thick sections, mounted onto poly L-lysine-coated slides, and dried. After the dried slides were de-paraffinized, antigen retrieval was performed using an automated antigen retrieval machine for 20 min using ethylenediaminetetraacetic acid (pH 9.0). Non-specific binding to the sections was blocked via incubation for one hour in 15% to 20% normal goat serum (Gibco Life Technologies, NY, USA) prior to incubation with an appropriate primary antibody for two hour at 22 to 25° C. or overnight at 4° C. A secondary rabbit antibody was used to detect the primary antibody, and then detection was performed using streptavidin-tagged horseradish peroxidase (Ventana Medical Systems, Tucson, AZ, USA). Diaminobenzidine (Sigma-Aldrich) was used to induce signaling, and a bluing reagent (Ventana Medical Systems) was used as a counterstain. The IHC slides were visualized using an optical microscope (Leica) and rendered using Leica software. IHC staining of the antibody against PCNA (Cat. No. sc-56) was performed.

### 14. ROS Assay

After the male rats were euthanized, the prostate tissues were excised and treated with type I collagenase (Thermo Fisher Scientific, MA, USA) to isolate the cells. Single cells from primary prostate tissues were mixed with the Muse® Oxidative Stress Reagent working solution. The cells were incubated for 30 minutes at 37° C. and detected using a Muse® Cell Analyzer (Merck, Darmstadt, Germany).

### 15. DHT and Testosterone Measurement

The serum DHT and testosterone levels were quantified using commercial ELISA kits (Cusabio, TX, USA). The assays were performed according to the manufacturer's instructions.

### 16. Statistical Analysis

Experiments were performed in triplicate, and the results are expressed as mean±standard deviation (SD). Statistically significant values were determined using analysis of variance and Dunnett's post-hoc test. Statistical significance was set at p<0.05. The statistical analysis was performed using GraphPad Prism 5.

### Results

1. MitoQ suppressed prostate cell proliferation and suppressed oxidative stress in DHT-stimulated RWPE-1 cells.

Since BPH is based on abnormal proliferation of prostate cells, regulating their proliferation is considered effective for BPH. RWPE-1 cell proliferation was suppressed by 25 μM and higher concentrations of MitoQ (FIG. 1B). It was found that under DHT stimulation, RWPE-1 cells proliferation increased, but the proliferation was significantly suppressed by treatment with 6.25 μM or higher concentrations of MitoQ (FIG. 1C). The antiproliferative effects of MitoQ were evaluated at concentrations of 25, 50, and 100 μM. Next, overexpression of 8OHdG, an indicator of oxidative stress, was observed in the DHT-stimulated RWPE-1 cells, but the overexpression was notably suppressed by MitoQ treatment (FIG. 1D). In addition, the mRNA level of HO-1 and GPX-1 was significantly lower in the DHT group than in the vehicle group.

The MitoQ treatment normalized these levels, and 100 μM MitoQ significantly increased the mRNA level of HO-1 and GPX-1, suggesting its antioxidant effects in the DHT-stimulated prostate cells (FIG. 1E). DHT stimulation also induced excessive production of mtROS through H₂O₂ in the RWPE-1 cells, but the excessive production was suppressed by the MitoQ treatment (FIG. 1F). To regulate oxidative stress created by mtROS, mitochondria operate an ROS-scavenging system, where superoxide dismutase (SOD) converts superoxide radicals into H₂O₂, which is further detoxified by catalase.

As shown in FIG. 1G, MitoQ treatment significantly increased SOD2 and catalase protein expression in the DHT-stimulated RWPE-1 cells, exhibiting an antioxidant effect on mtROS in the androgen-stimulated prostate cells.

### 2. MitoQ Exerted Antioxidant Effects by Inhibiting NLRP3 in DHT-Stimulated RWPE-1 Cells.

The NLRP3 inflammasome was studied to investigate the signaling pathway underlying MitoQ suppression of prostate cell proliferation.

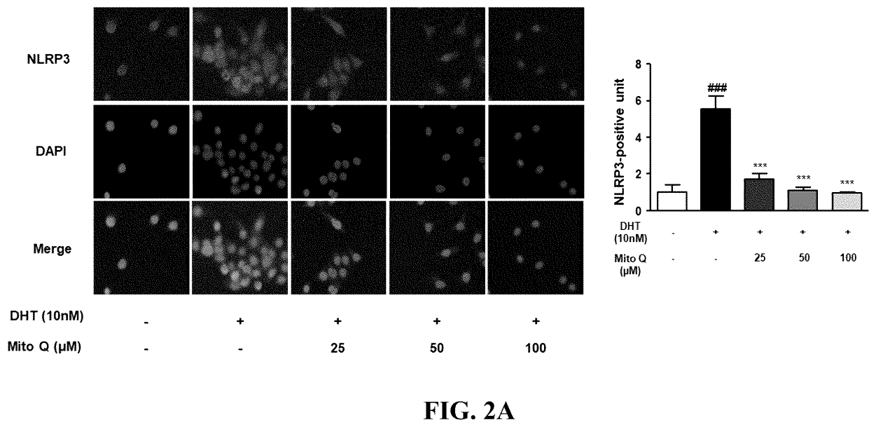

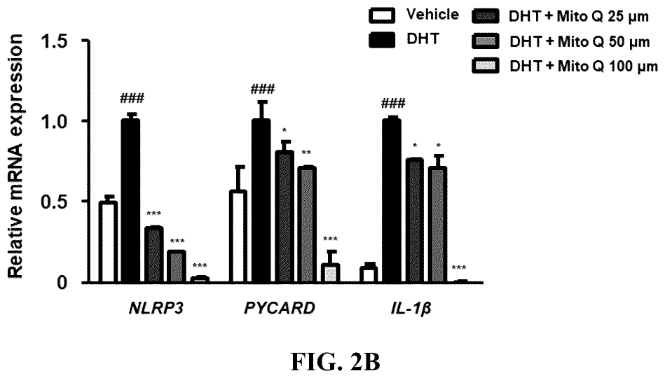

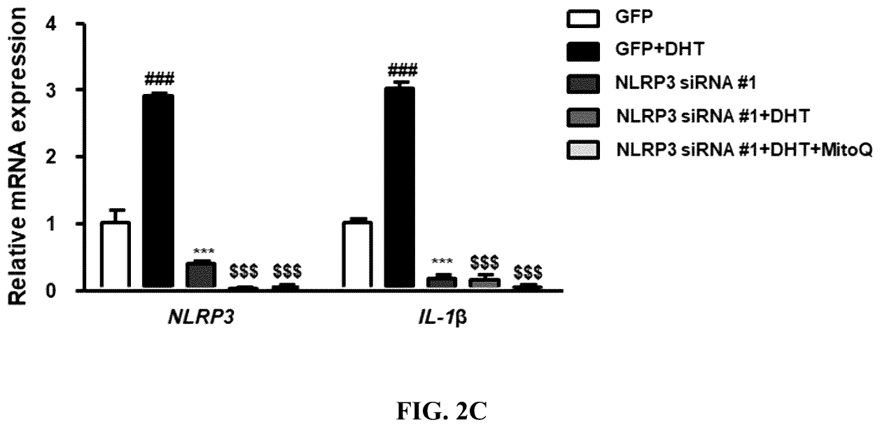

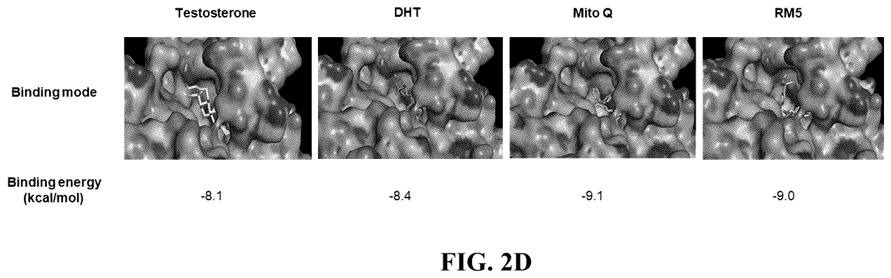

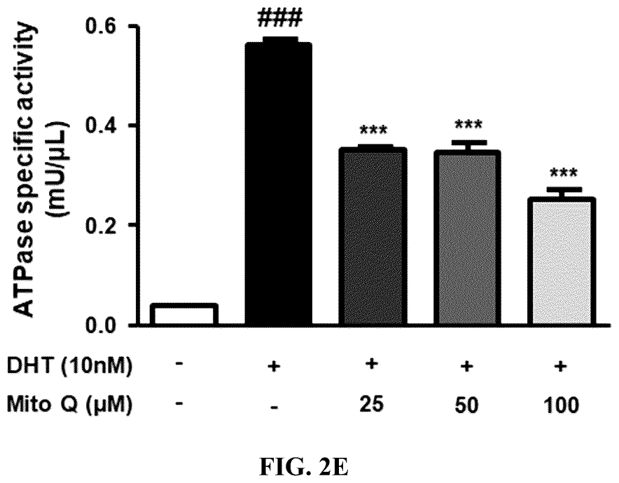

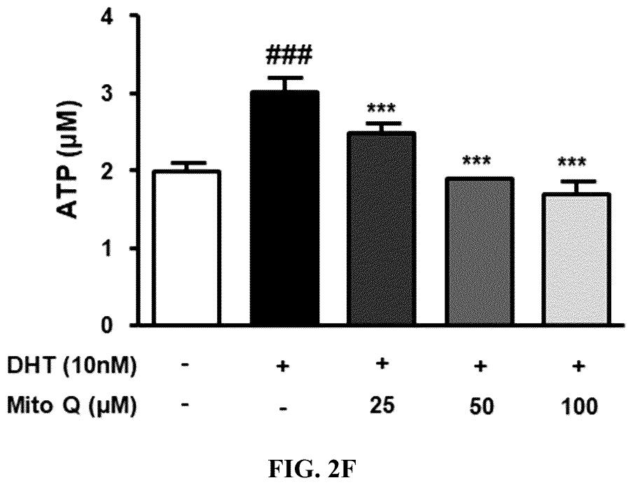

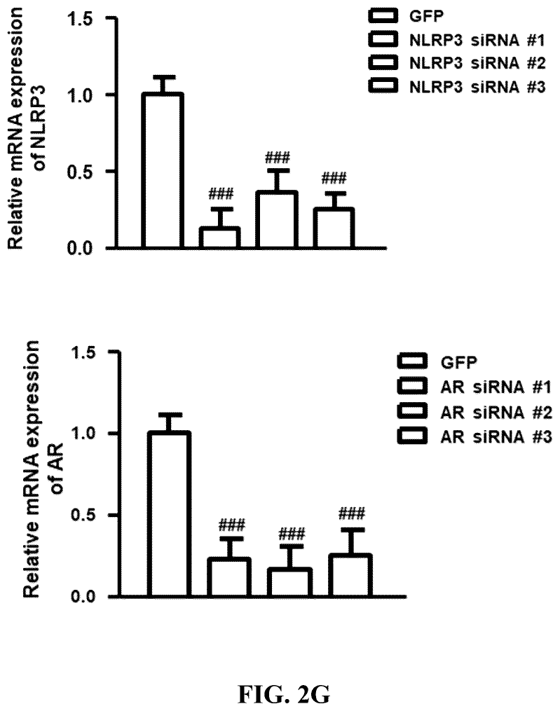

Since MitoQ inhibits mtROS by repressing the NLRP3 inflammasome, it was assumed MitoQ inhibits NLRP3 expression in DHT-stimulated RWPE-1 cells. NLRP3 protein expression increased under DHT stimulation of the RWPE-1 cells, whereas MitoQ significantly suppressed it (FIG. 2A). In addition, the mRNA expression of NLRP3, PYCARD, and IL-1β was significantly increased in the DHT-stimulated RWPE-1 cells, whereas it was significantly reduced by the MitoQ treatment (FIG. 2B). To clarify the effect of MitoQ on NLRP3 signaling, the RWPE-1 cells were transfected with NLRP3 siRNA and then stimulated with DHT. The significant decrease in NLRP3 mRNA expression in the RWPE-1 cells was confirmed using NLRP3 siRNA (FIG. 2G). In green fluorescent protein (GFP)-transfected cells, the DHT stimulation significantly increased the NLRP3 and IL-1β levels. In contrast, NLRP3 siRNA transfection notably suppressed their levels and neutralized the effect of MitoQ on the RWPE-1 cells (FIG. 2C). As shown in FIG. 2D, a molecular docking simulation was performed to validate whether MitoQ could directly bind to the NACHT domain of NLRP3. MitoQ, testosterone, DHT, and RM5, which is a recently identified inhibitor, were docked into the NACHT domain. MitoQ showed an affinity value of −9.1 kcal/mol, and testosterone and DHT showed higher binding energy values (−8.1 and −8.4 kcal/mol, respectively). In addition, RM5 showed an even higher binding energy of −9.0 kcal/mol. Of the four ligands, MitoQ was predicted to be the strongest binding ligand (lowest binding energy=−9.1 kcal/mol). Since the 3D binding structure of RM5 onto the NACHT domain was determined using X-ray crystallography, the better binding affinity of MitoQ predicted via the docking simulation implies that MitoQ is also a strong binding ligand to the NACHT domain of NLRP3.

The ATPase activity is involved in NLRP3 inflammasome activation, so the inhibitory effect of MitoQ on the ATPase activity in the DHT-stimulated RWPE-1 cells was estimated (FIG. 2E). The ATPase activity was significantly higher in the DHT group than that in the vehicle group, whereas the MitoQ treatment significantly suppressed the ATPase-specific activity. In addition, the ATP level of the DHT-stimulated RWPE-1 cells was analyzed.

The DHT stimulation significantly increased the ATP level, whereas MitoQ treatment significantly reduced it in a dose-dependent manner (FIG. 2F).

### 3. MitoQ Suppressed the AR Signaling Pathway in DHT-Stimulated RWPE-1 Cells.

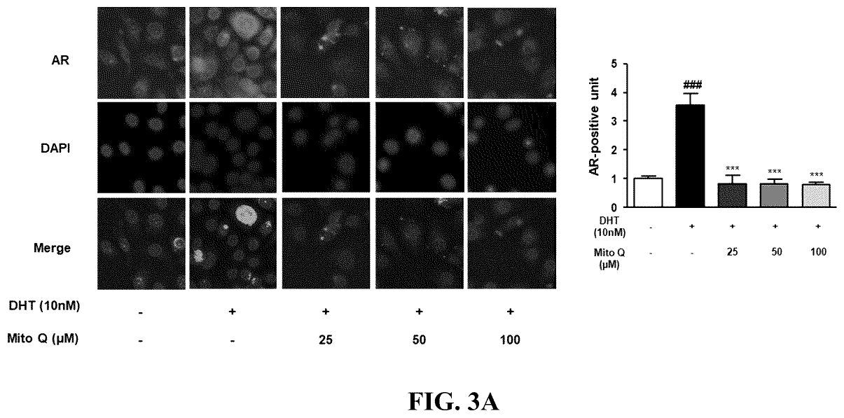

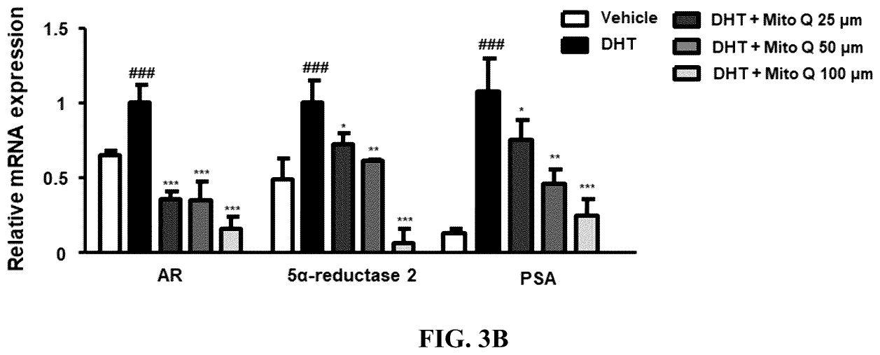

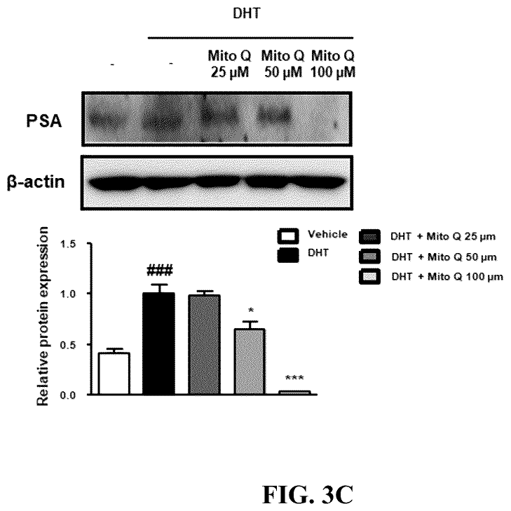

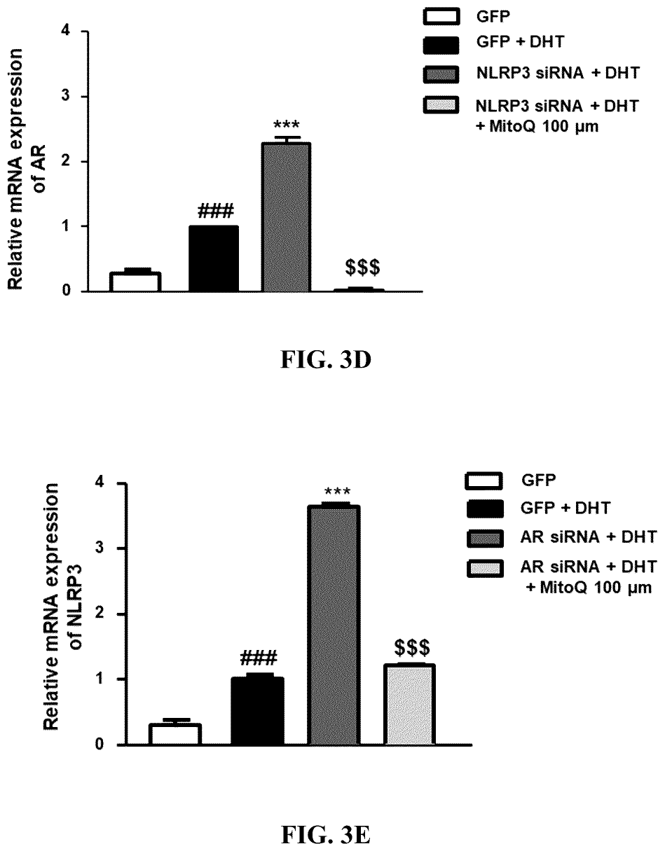

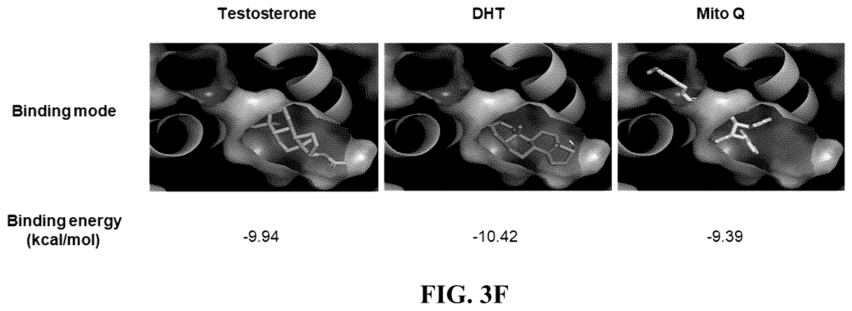

BPH is based on androgen-dependent abnormal proliferation of prostate cells, so regulating androgen/AR signaling is considered effective in BPH. As shown in FIG. 3A, the DHT stimulation induced AR overexpression in the RWPE-1 cells, and the AR overexpression was clearly suppressed by MitoQ treatment. In addition, the mRNA level of AR, 5α-reductase 2, and PSA was significantly enhanced in the DHT-stimulated group, whereas MitoQ treatment notably inhibited it (FIG. 3B). The DHT stimulation also increased PSA protein expression, which was considerably suppressed by MitoQ (FIG. 3C). The AR contributes to testosterone-induced NLRP3 inflammasome activation and oxidative stress. The contribution of NLRP3 and AR signaling to the effect of MitoQ on DHT-stimulated prostate cells was further evaluated using siRNA. As shown in FIG. 3D, the DHT stimulation significantly increased the AR level in the GFP-transfected cells. Interestingly, NLRP3 siRNA transfection also significantly increased the AR level under the DHT stimulation, but the AR level was notably downregulated by the MitoQ treatment. Furthermore, the DHT stimulation significantly increased the NLRP3 level in the GFP-transfected cells. The AR siRNA transfection also significantly enhanced the NLRP3 level, whereas the MitoQ treatment significantly suppressed them (FIG. 3E). These results suggest that DHT may bind to AR and NLRP3 to increase their expression and that MitoQ serves as an inhibitor of both AR and NLRP3. Next, direct binding of MitoQ to AR was investigated through molecular docking. The docking was performed using a DHT-bound AR structure. To compare the docking affinity values of the ligands, the DHT already bound to this structure was removed and then re-docked. The DHT was docked to the same binding site, which was very similar to the shape shown by X-ray crystallography, and exhibited an affinity value of −10.42 kcal/mol. Testosterone and MitoQ were confirmed to bind to the binding site, and their predicted affinity values in the docking simulation were −9.94 and −9.39 kcal/mol, respectively (FIG. 3F). Although the predicted MitoQ binding was not stronger than that of testosterone and DHT, it seemed that MitoQ was bound to AR, because the difference between the values of DHT and MitoQ was very small (˜1 kcal/mol).

### 4. MitoQ Alleviated Prostate Enlargement and Prostate Cell Proliferation in Rats With BPH.

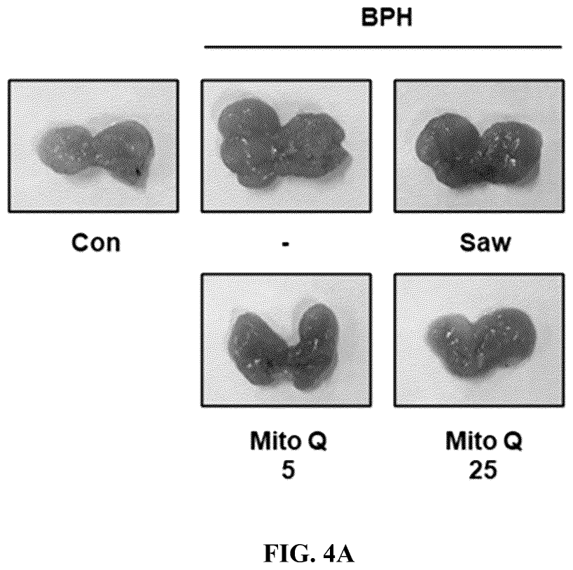

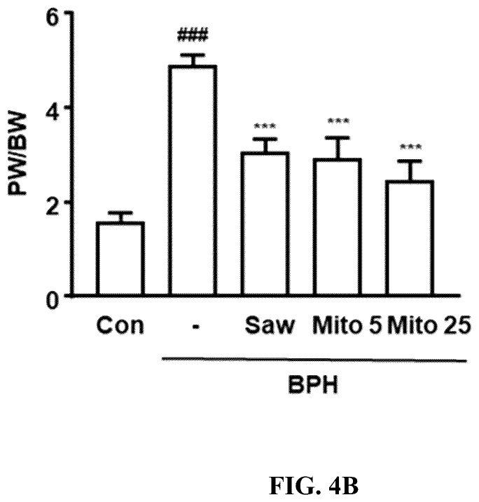

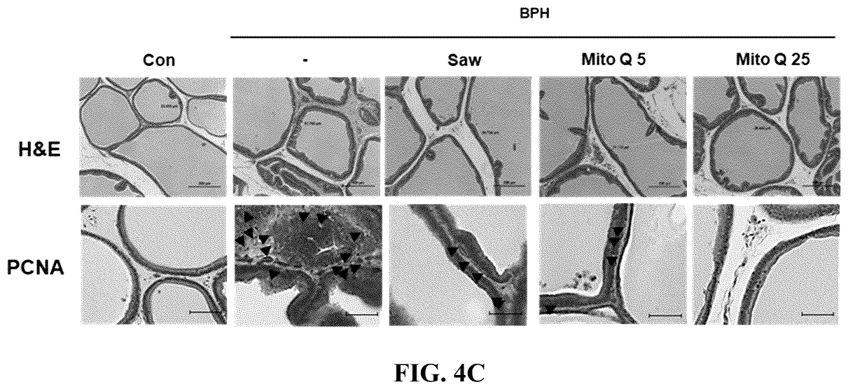

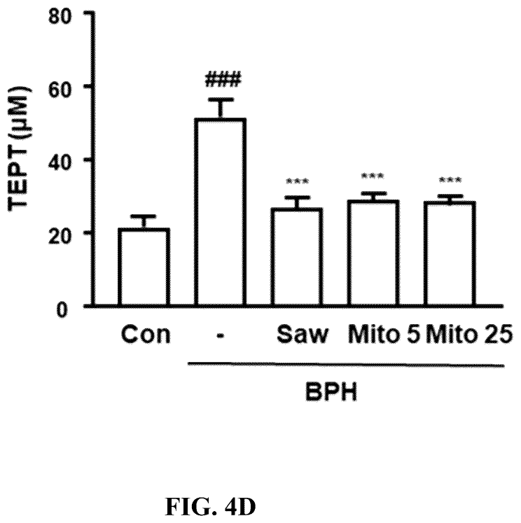

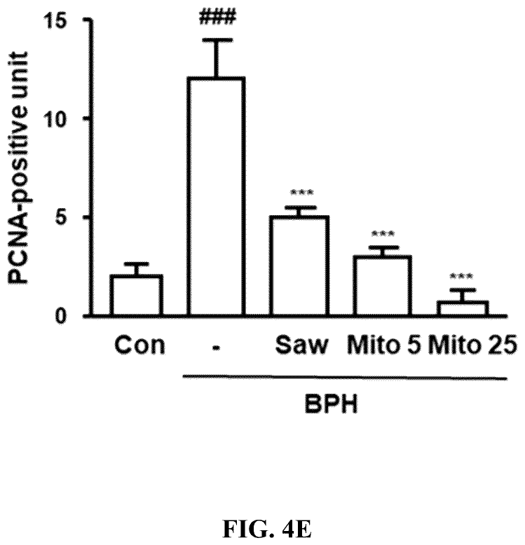

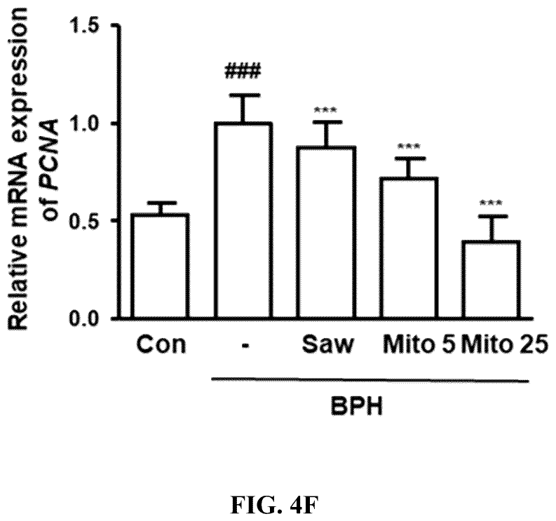

In consideration of the suppressive effects of MitoQ on DHT-dependent prostate cell proliferation in vitro, its therapeutic effects were evaluated in a testosterone-induced BPH rat model. Testosterone injection resulted in prostate enlargement. Saw, a highly effective anti-androgen, was used as a positive control in the experimental system of the present invention. The administration of 5 and 25 mg/kg of MitoQ reduced the prostate size in the positive control (FIG. 4A). Rats with BPH exhibited increased prostate weight, whereas rats in the Mito and Saw groups showed a significant reduction in the prostate weight (FIG. 4B). A histological analysis showed a hyperplastic pattern in the BPH groups, including thickened epithelium, whereas the administration of Saw and MitoQ restored the histological changes and significantly reduced the thickened epithelium (FIGS. 4C and 4D). In addition, the effect of MitoQ on the cell proliferation marker PCNA was analyzed. The BPH groups showed significantly overexpressed PCNA protein and mRNA compared to the Con group, whereas the administration of Saw and MitoQ clearly suppressed such overexpression (FIGS. 4C, 4E, and 4F). In particular, the cellular proliferation inhibiting effect of MitoQ 25 was superior to that of Saw.

### 5. MitoQ Exerted Antioxidant Effects in Rats With BPH.

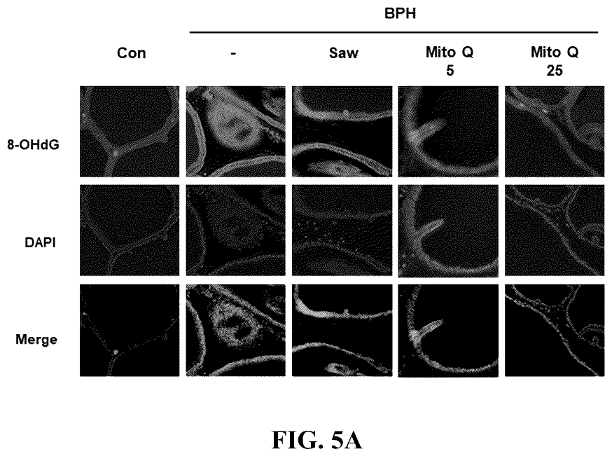

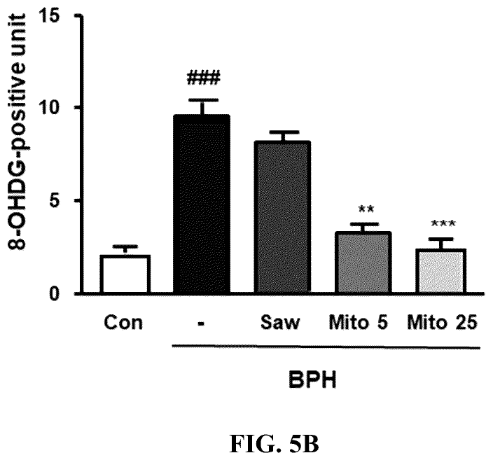

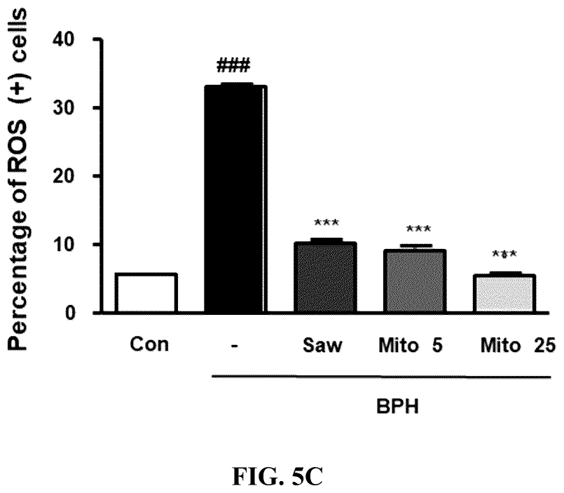

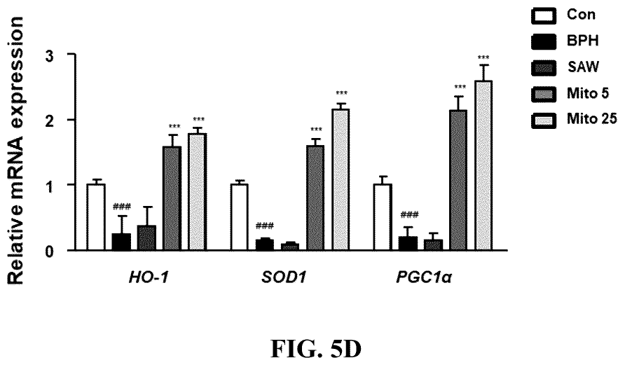

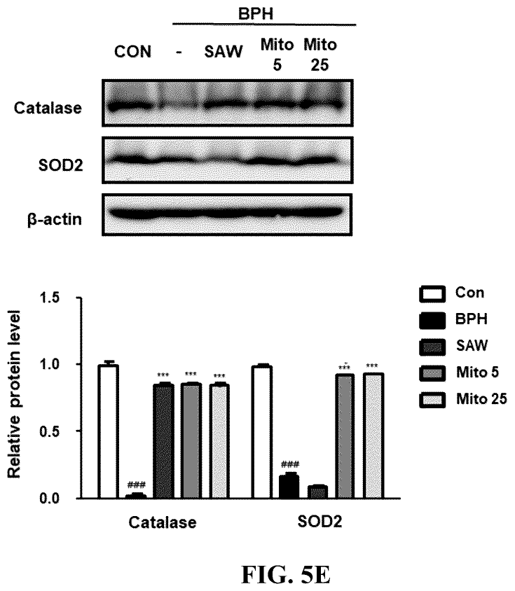

Early studies have proved that androgen deprivation therapy may decrease oxidative stress. Therefore, the expression of 8-OHdG, an oxidative stress indicator, was examined in the prostate tissues from a testosterone-induced BPH rat model. The intensity of 8-OHdG-positive staining in the BPH groups was significantly higher than that in the Con group. On the contrary, this intensity was significantly lower in the MitoQ-treated group than that in the BPH groups (FIGS. 5A and 5B). To identify whether MitoQ exerts an antioxidant effect in the testosterone-induced BPH rat model, the extent of ROS production and the levels of antioxidant enzymes in the prostate tissues were assessed.

As shown in FIG. 5C, the BPH groups induced significantly more generation of intracellular ROS compared to the Con group, whereas the administration of MitoQ significantly suppressed ROS generation in a dose-dependent manner. In addition, the mRNA level of HO-1, SOD1, and PGC1α was lower in the BPH groups than in the Con group. On the contrary, the level was upregulated by the MitoQ administration, but not by Saw (FIG. 5D). As shown in FIG. 5E, the expression of catalase and SOD2 was significantly lower in the BPH groups than in the Con group. In contrast, the expression was significantly higher in the MitoQ-treated group than in the BPH groups.

### 6. MitoQ Inhibited the NLRP3 Signaling Pathway in Rats With BPH.

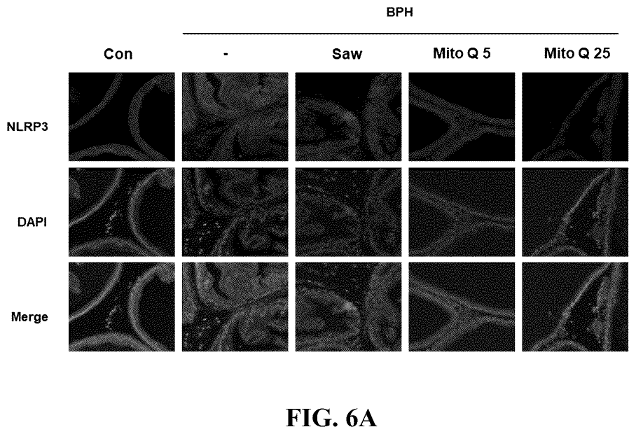

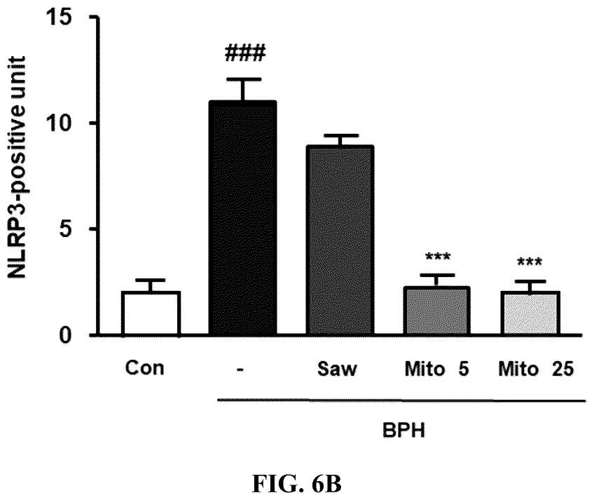

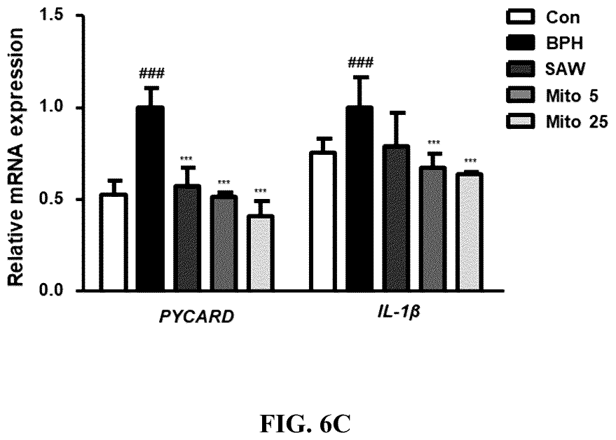

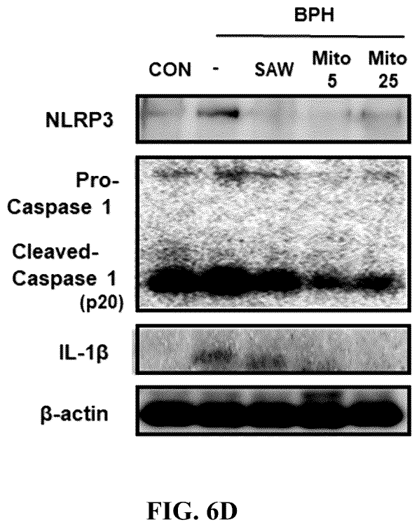

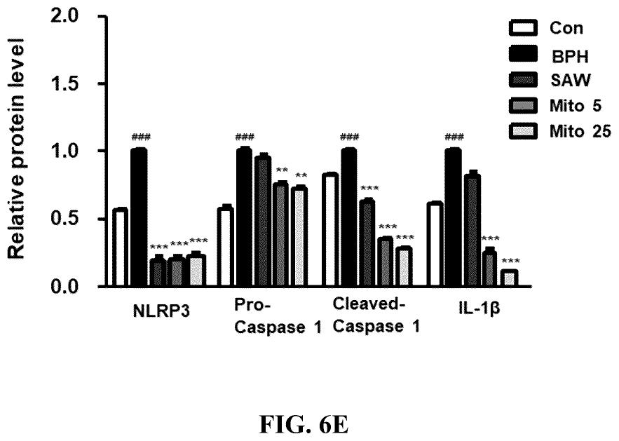

Next, the involvement of the NLRP3 inflammasome in the prostate tissues from rats with BPH was analyzed. BPH upregulated the expression of NLRP3, which was reversed by MitoQ treatment (FIGS. 6A and 6B).

FIG. 6C also shows that the increased mRNA level of PYCARD and IL-1β induced by BPH was repressed by the MitoQ administration. As shown in FIGS. 6D and 6E, the BPH induction increases the protein expression of NLRP3, pro-caspase 1, cleaved caspase 1, and IL-1β, compared to the Con group. However, the MitoQ treatment suppressed the overexpression of these proteins. These results strongly support the protective effects of MitoQ in BPH through the regulation of the NLRP3 signaling pathway.

### 7. MitoQ Repressed the Expression of Androgen-Relative Markers in Rats With BPH.

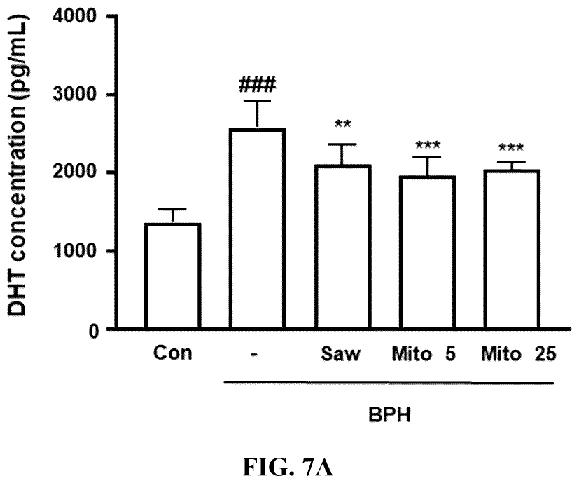

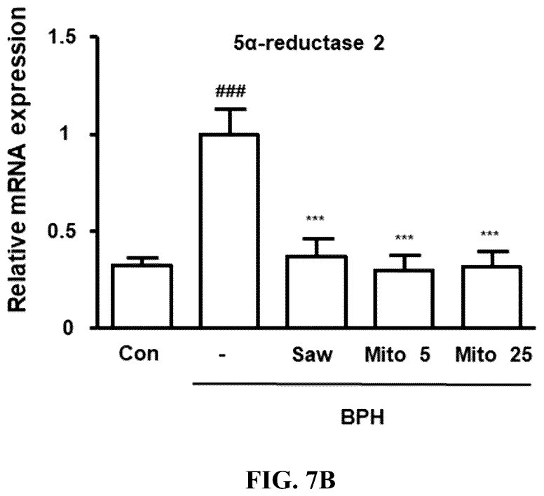

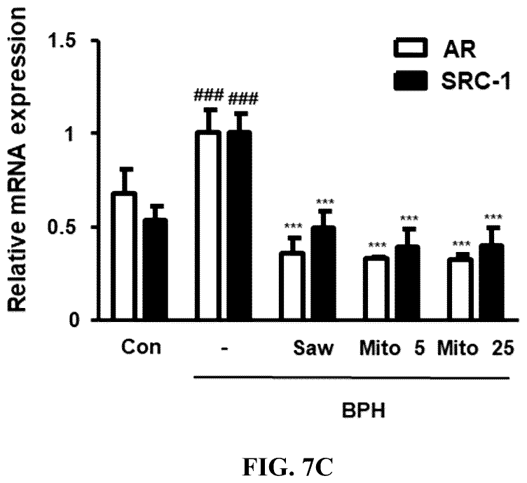

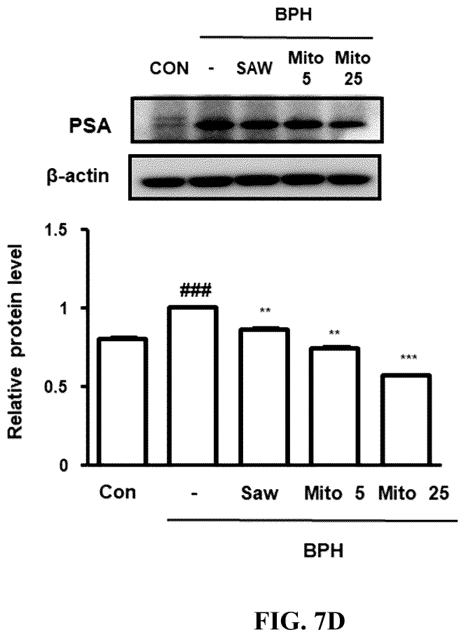

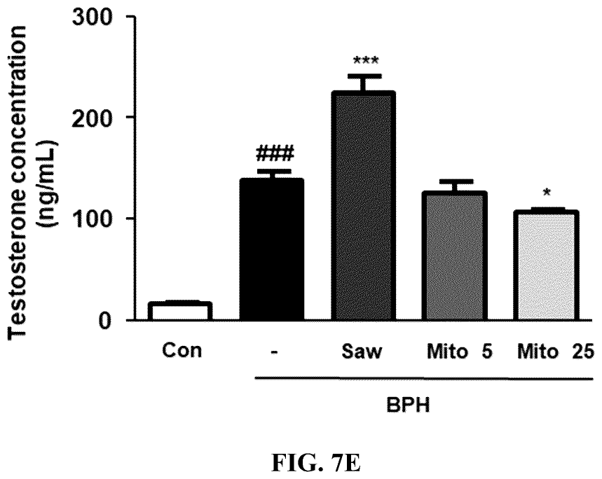

Considering the in vitro results where the MitoQ treatment suppressed AR and 5α-reductase levels, its inhibitory effects on androgen signaling were investigated in rats with BPH. As shown in FIG. 7A, the BPH induction increased DHT production, whereas MitoQ and Saw administration significantly suppressed the overproduction. In addition, the BPH induction significantly increased the circulating testosterone level, which was significantly reduced by 25 mg/kg MitoQ. The Saw administration increased the serum testosterone level in BPH rats (FIG. 7E). As shown in FIGS. 7B and 7C, the Saw and MitoQ administration downregulated the mRNA level of 5α-reductase 2, AR, and SRC-1, which was enhanced by the BPH induction. The protein expression of PSA, a biomarker for the diagnosis of prostate diseases, increased in the BPH groups and decreased in Saw and MitoQ (FIG. 7D). These findings suggest that MitoQ suppresses BPH progression through androgen signaling.

The present invention relates to a pharmaceutical composition and a health functional food for preventing or treating BPH. The present invention includes mitoquinone as an active ingredient and inhibits the AR and NLRP3 signaling pathways to alleviate pathological prostatic hypertrophy and exhibit anti-proliferative and antioxidant effects. Therefore, it can be effectively used in preventing, treating, or ameliorating BPH.

The effects of the present invention are not limited to the above-described effects and should be understood to include all effects that may be inferred from the configuration of the invention described in the description or claims of the present invention.

What is claimed is:

1. A pharmaceutical composition for preventing or treating benign prostatic hyperplasia (BPH), comprising mitoquinone or a pharmaceutically acceptable salt thereof.

2. The pharmaceutical composition of claim 1, wherein the mitoquinone is included at 25 μM or more.

3. The pharmaceutical composition of claim 1, wherein the mitoquinone inhibits androgen receptor (AR) and nucleotide oligomerization domain (NOD)-like receptor family pyrin domain-containing 3 (NLRP3) signaling pathways.

4. The pharmaceutical composition of claim 1, wherein the mitoquinone inhibits dihydrotestosterone (DHT)-induced prostate cell proliferation.

5. A health functional food for preventing or ameliorating benign prostatic hyperplasia (BPH), comprising mitoquinone or a stiologically acceptable salt thereof.

6. The health functional food of claim 5, wherein the mitoquinone is included at 25 μM or more.

7. The health functional food of claim 5, wherein the mitoquinone inhibits androgen receptor (AR) and nucleotide oligomerization domain (NOD)-like receptor family pyrin domain-containing 3 (NLRP3) signaling pathways.

8. The health functional food of claim 5, wherein the mitoquinone inhibits dihydrotestosterone (DHT)-induced prostate cell proliferation.

9. A method of treating benign prostatic hyperplasia (BPH), the method comprising administering to a patient in need of treatment mitoquinone, a pharmaceutically acceptable salt thereof, or a pharmaceutical composition including mitoquinone.

10. The method of claim 9, wherein the mitoquinone is included at 25 μM or more.

11. The method of claim 9, wherein the mitoquinone inhibits androgen receptor (AR) and nucleotide oligomerization domain (NOD)-like receptor family pyrin domain-containing 3 (NLRP3) signaling pathways.

12. The method of claim 9, wherein the mitoquinone inhibits dihydrotestosterone (DHT)-induced prostate cell proliferation.

13. The method of claim 9, wherein the administering is performed parenterally or orally.
